(19) United States
(12) Patent Application Publication
(10) Pub. No.: US 2008/0199504 A1
(43) Pub. Date: Aug. 21, 2008

(54) Dynamers for therapeutic agent delivery applications

(76) Inventors: Syed Faiyaz Ahmed Hossainy, Fremont, CA (US); Florian Ludwig, Mountain View, CA (US); Mikael Trollsas, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/707,791

(22) Filed: Feb. 15, 2007

(51) Int. Cl. A61F 2/00; A61K 47/00; A61P 9/00

(52) U.S. Cl. 424/423; 514/772.3

(57) ABSTRACT

Dynamers for use in therapeutic agent delivery systems are disclosed.

### FIELD OF THE INVENTION

The present invention relates to dynamers for therapeutic agent delivery applications.

### BACKGROUND OF THE INVENTION

The traditional method of administering therapeutic agents to treat diseases of the internal organs and vasculature has been by systemic delivery. Systemic delivery involves administering a therapeutic agent at a discrete location followed by the agent migrating throughout the patient's body including, of course, to the afflicted organ or area of the vasculature. But to achieve a therapeutic amount of the agent at the afflicted site, an initial dose substantially greater than the therapeutic amount must be administered to account for the dilution the agent undergoes as it travels through the body. Systemic delivery introduces the therapeutic agent in two ways: into the digestive tract (enteral administration) or into the vascular system (parenteral administration), either directly, such as injection into a vein or an artery, or indirectly, such as injection into a muscle or into the bone marrow. Absorption, distribution, metabolism, excretion and toxicity, the ADMET factors, strongly influence delivery by each of these routes. For enteric administration, factors such as a compound's solubility, its stability in the acidic environs of the stomach and its ability to permeate the intestinal wall all affect drug absorbtion and therefore its bioavailability. For parenteral delivery, factors such as enzymatic degradation, lipophilic/hydrophilic partitioning coefficient, lifetime in circulation, protein binding, etc. will affect the agent's bioavailability.

At the other end of the spectrum is local delivery, which comprises administering the therapeutic agent directly to the afflicted site. With localized delivery, the ADMET factors tend to be less important than with systemic administration because administration is essentially directly to the treatment site. Thus, the initial dose can be at or very close to the therapeutic amount. With time, some of the locally delivered therapeutic agent may diffuse over a wider region, but that is not the intent of localized delivery, and the diffused portion's concentration will ordinarily be sub-therapeutic, i.e., too low to have a therapeutic effect. Nevertheless, localized delivery of therapeutic agents is currently considered a state-of-the-art approach to the treatment of many diseases such as, without limitation, cancer and atherosclerosis.

Localized delivery of therapeutic agents includes the targeted delivery of therapeutic agent-containing compositions. This method can consist of administering a composition containing a therapeutic agent directly to a disease locale, e.g., injecting the composition into the vasculature at or near the disease site.

Localized therapeutic agent delivery also includes using implantable medical devices, e.g., stents. By positioning a therapeutic-agent-coated stent at a target site, agents can be applied directly to the lumen area requiring therapy.

Both of these methods depend on the controlled release of the therapeutic agent, which is primarily governed by the choice of polymer used to coat the device or used to form the agent-containing components of the compositions.

There is, therefore, an ongoing need for novel polymeric materials that provide more effective and tunable mechanisms for delivering therapeutic agents to a patient.

The present invention provides molecular, super-molecular and supra-molecular assemblies for use in therapeutic agent delivery systems, as well as methods of using these systems for treating disease.

### SUMMARY OF THE INVENTION

The present invention relates to an implantable medical device that includes a dynamer. The dynamer includes covalent bonds that are reversible under physiological conditions, and the implantable medical device can be a stent.

In various aspects, the reversible covalent bonds include hydrazones, imines, oximes, or a combination thereof.

In various aspects, the device includes a therapeutic agent, which can be an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, and ABC Al agonist or an antioxidant.

The dynamer can further exhibit reversible non-covalent interactions. The reversible non-covalent interactions can be reversible under physiological conditions and include hydrogen bonding, ionic bonding, chelation, charge-transfer complexes, pi-stacking, hydrophobic interactions, electrostatic interactions, magnetic interactions and van der Waals forces.

Another aspect of the present invention relates to a coating for a medical device that includes a dynamer. The dynamer includes covalent bonds that are reversible under physiological conditions.

In various aspects, the reversible covalent bonds include hydrazones, imines, oximes, or a combination thereof.

The dynamer can further exhibit reversible non-covalent interactions. The reversible non-covalent interactions are reversible under physiological conditions and include hydrogen bonding, ionic bonding, chelation, charge-transfer complexes, pi-stacking, hydrophobic interactions, electrostatic interactions, magnetic interactions and van der Waals forces.

In various aspects, the coating can be used as a primer layer, a reservoir layer, a topcoat layer, or any combination thereof.

Another aspect of the present invention relates to a method of treating or preventing a vascular disease. The method involves providing an implantable medical device of the present invention and implanting the medical device in a vessel of a patient in need thereof.

In various aspects, the vascular disease can be atherosclerosis, restenosis, vulnerable plaque or peripheral arterial disease.

Another aspect of the present invention relates to a composition that includes a dynamer and a therapeutic agent encapsulated or embedded within the dynamer. The dynamer includes covalent bonds that are reversible under physiological conditions.

In various aspects, the reversible covalent bonds include hydrazones, imines, oxime, or a combination thereof.

In various aspects, the dynamer includes particles, hydrogel particles, a polymer depot or a hydrogel depot.

The therapeutic agent can be an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, and ABC Al agonist or an antioxidant.

In an aspect of the present invention, the dynamer further exhibits reversible non-covalent interactions. The reversible non-covalent interactions are reversible under physiological conditions and include hydrogen bonding, ionic bonding, chelation, charge-transfer complexes, pi-stacking, hydrophobic interactions, electrostatic interactions, magnetic interactions and van der Waals forces.

Another aspect of the present invention relates to a method that involves providing a composition of the present invention and administering a therapeutically effective amount of the composition to a disease locale in a patient. The disease locale can be a vascular disease locale.

In various aspects, administering the composition to the disease locale can include intravenous, intraarterial, intraadventitial, intraperiadvential, intramyocardial, subcutaneous, intramuscular, intra-organ, intra-tumor, or subxyphoid delivery of the composition.

In one embodiment, administering the composition to the disease locale includes using a device. The device can be a catheter, an implantable medical device or a stent.

In various aspects, the vascular disease can be atherosclerosis, restenosis, vulnerable plaque or peripheral arterial disease.

### BRIEF DESCRIPTION OF THE DRAWINGS

FIG. 1 is a schematic illustration of an acylhydrazone depicting potential hydrogen bonding sites and a reversible C═N moiety.

FIG. 2 is a representation of a supra-molecular dynamer illustrating reversible covalent bonding sites and potential non-covalent interaction sites between groups on the same dynamer.

FIG. 3 is a representation of a super-molecular dynamer illustrating reversible covalent bonding sites and potential non-covalent interaction sites between groups on different dynamers.

### DETAILED DESCRIPTION OF THE INVENTION

### Definitions:

As used herein, an “implantable medical device” refers to any type of appliance that is totally or partly introduced, surgically or medically, into a patient's body or by medical intervention into a natural orifice. The duration of implantation may be essentially permanent, i.e., intended to remain in place for the lifespan of the patient; until the device biodegrades; or until it is physically removed. Presently preferred implantable medical devices include, without limitation, catheters, and more preferably stents. Stents can be self-expandable stents or balloon-expandable stents. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or a combination thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention.

As used herein, “polymer” refers to a molecule(s) composed of a plurality of repeating structural units connected by covalent chemical bonds.

As used herein, “covalent bonding” refers to an interaction between two atoms in which in the ground, i.e., non-excited, state the atoms share one pair of electrons, i.e., one electron from each atom, two pairs of electrons, i.e., two electrons from each atom, or three pairs of electrons, i.e., three electrons from each atom, to form a single, double or triple covalent bond as such is known and understood by those of ordinary skill in the synthetic organic chemistry art.

As used herein, “reversible” refers to the property of both covalent bonds and non-covalent interactions to form and break repeatedly.

As used herein, “physiological conditions” refer to the physical or chemical environment inside of a patient.

As used herein, “non-covalent interactions” refer to any interaction between atoms other than a covalent interaction, as defined herein. These non-covalent interactions include, without limitation, hydrogen bonding, ionic bonding, chelation, charge-transfer complexes, pi-stacking, hydrophobic interactions, electrostatic interactions, magnetic interactions and van der Waals forces.

As used herein, “hydrogen bonding” refers to the interaction between a hydrogen atom that is attached to a relatively electronegative element, e.g., oxygen or nitrogen, and a lone pair of electrons on another atom.

As used herein, “ionic bonding” refers to reversible bonding based on electrostatic forces between two oppositely-charged ions, i.e., negative and positive ions.

As used herein, “chelation” refers to the reversible process of binding of a chelator, e.g., porphyrin, to a metal ion, e.g., Fe⁺², to form a complex called a chelate, e.g., heme.

As used herein, a “charge-transfer complex” refers to an electron donor-electron acceptor complex characterized by electronic transitions to an excited state. When in this excited state, there is a partial transfer of electronic charge from the donor to the acceptor. The formation of these complexes is readily reversible.

As used herein, “pi-stacking”, i.e., π-π interaction, refers to a reversible noncovalent interaction between aromatic organic compounds caused by intermolecular overlapping of their delocalized p-orbitals.

As used herein, “hydrophobic interactions” refer to reversible attractive interactions between nonpolar molecules in a polar environment.

As used herein, “electrostatic interactions” refer broadly to interactions between charged species.

As used herein, “magnetic interactions” refer broadly to attractive or repulsive forces between two or more materials due to the magnetic properties of the respective materials. Incorporating magnetized particulates into a molecule of the invention will allow for magnetic interactions.

As used herein, “van der Waals interactions” refer to reversible interactions stemming from the polarization of molecules into dipoles, i.e., unequal distribution of electronic charge within a molecule. The transient polarization of the molecules allows for reversible partial positive ion and partial negative ion electrostatic interactions.

As used herein, “dynamer” refers to materials whose chemical constituents are linked through reversible connections and are able to undergo continuous reorganization through assembly/disassembly processes, incorporation, or reshuffling of constituents under a given set of conditions, e.g., under thermodynamic control. Dynamers exhibit reversible covalent bonding and have the potential to exhibit reversible noncovalent interactions, e.g., supra- and super-molecular interactions.

As used herein, “supra-molecular” interactions refer to covalent and noncovalent interactions between chemical groups present on the same molecule.

As used herein, “super-molecular” interactions refer to covalent and noncovalent interactions between chemical groups on different molecules.

As used herein, “hydrazone” refers to a class of organic compounds formed by the reaction of a hydrazide derivative with an aldehyde or ketone, as shown in equation I.

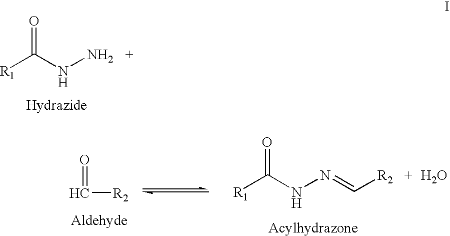

A key property of these compounds is the reversibility of the reactions, as exemplified by acylhydrazone formation that displays reversibility under mild conditions with acid catalysis.

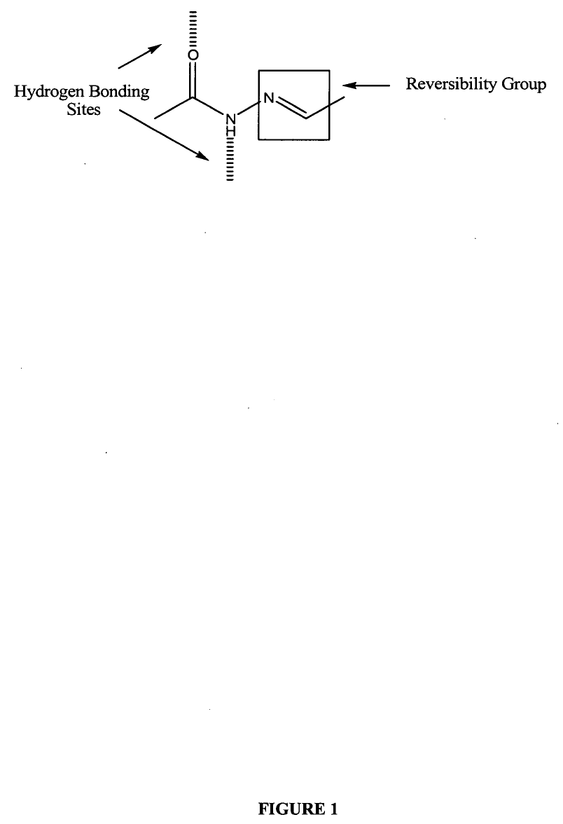

In the presence of additional aldehydes or hydrazides, the reversibility can be exploited to produce new acylhydrazones via aldehyde or hydrazide exchange promoted by acid catalysis and/or heat. In addition, the carbonyl groups and the hydrogen NH group provide hydrogen-bonding sites, as depicted in FIG. 1, thereby providing a second level of dynamic character, i.e., non-covalent interactions.

It is also possible to synthesize polyacylhydrazones by polycondensation involving dihydrazides and dicarbonyl compounds, as shown in equation II below.

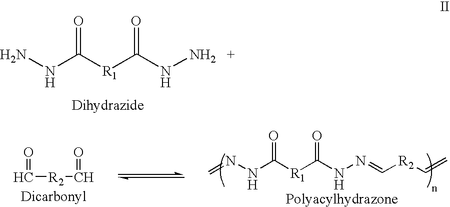

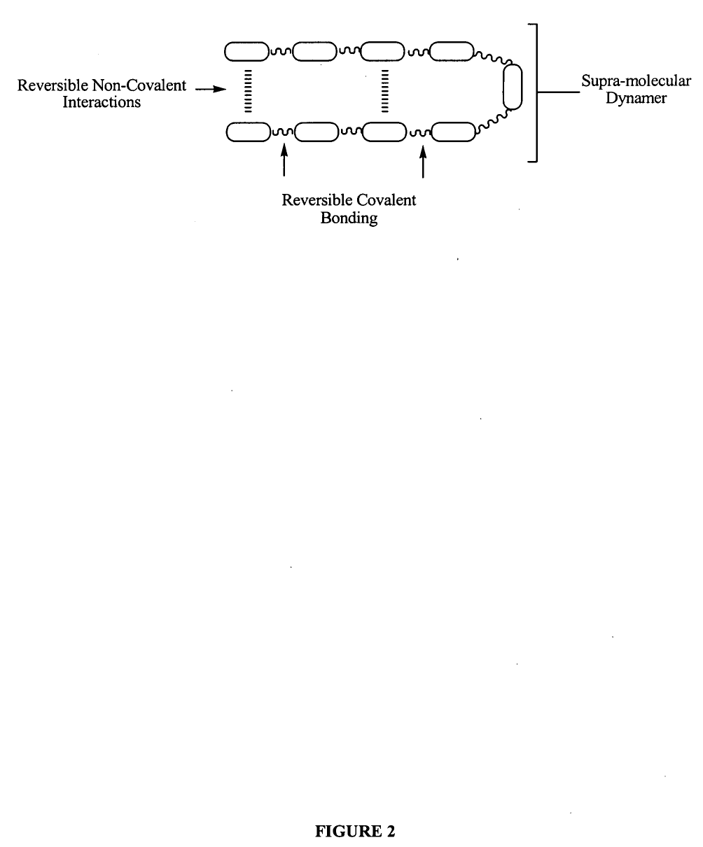

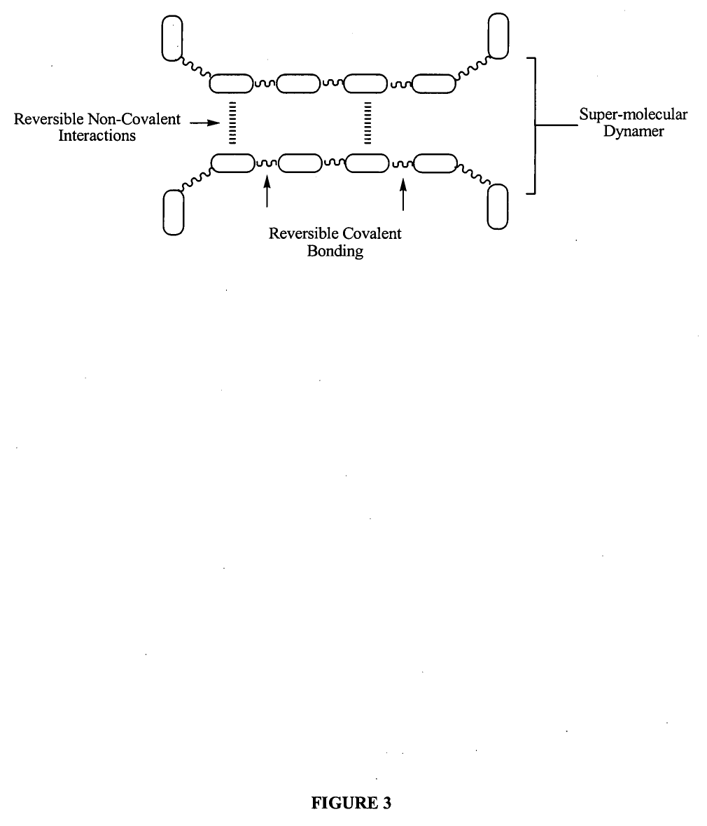

These polymers are not only capable of exchanging their components, but since they contain an amide group, supra- and super-molecular hydrogen bonding is possible as well, as represented schematically in FIGS. 2 and 3.

Other dynamers encompassed by the present invention include, but are not limited to, oximes and imines.

As used herein, “oxime” refers to a class of organic compounds formed by the reaction of an aldehyde or a ketone with hydroxylamine, and having the general structure

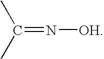

As used herein, “imine” refers generally to a class of organic compounds formed by reaction of a primary amine with an aldehyde or ketone, and having the general structure

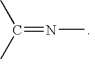

Similar to polyacylhydrazones, polymeric dynamer chains with oxime and imine backbones are also possible, and methods of preparing them are known to those skilled in the art. Furthermore, as with polyacylhydrazones, the reactions forming these dynamer chains are reversible and have the ability to exhibit noncovalent supra- and super-molecular interactions.

One aspect of the present invention relates to an implantable medical device that includes a dynamer either as an integral part of the structure of the device itself, or as a component of a coating applied to the device. When the dynamer is a component of a coating, the dynamer can form the coating itself or can be particles that are associated with the coating.

In one embodiment, the device further includes a therapeutic agent that can be an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC Al agonist or an antioxidant.

Suitable antiproliferative agents include, without limitation, actinomycin D, or derivatives or analogs thereof, i.e., actinomycin D is also known as dactinomycin, actinomycin IV, actinomycin I₁, actinomycin X₁, and actinomycin C₁. Antiproliferative agents can be natural proteineous agents such as a cytotoxin or a synthetic molecule, all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin, prodrugs thereof, co-drugs thereof, and combinations thereof.

Suitable anti-inflammatory agents include, without limitation, steroidal anti-inflammatory agents, a nonsteroidal anti-inflammatory agent, or a combination thereof. In some embodiments, anti-inflammatory agents include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. The anti-inflammatory agent may also be a biological inhibitor of proinflammatory signaling molecules including antibodies to such biological inflammatory signaling molecules.

Suitable antineoplastics and/or antimitotics include, without limitation, paclitaxel, docetaxel, methotrexate, azathioprine, vincristine, vinblastine, fluorouracil, doxorubicin hydrochloride, and mitomycin.

Suitable antiplatelet, anticoagulant, antifibrin, and antithrombin drugs include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä (Biogen, Inc., Cambridge, Mass.), calcium channel blockers (such as nifedipine), colchicine, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents that may be appropriate include alpha-interferon, and genetically engineered epithelial cells.

Suitable cytostatic or antiproliferative agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Suitable antiallergic agents include, without limitation, permirolast potassium.

Other suitable bioactive agents include, without limitation, alpha-interferon, genetically engineered epithelial cells, dexamethasone and its derivatives, rapamycin derivatives and analogs such as 40-O-(2-hydroxyethyl)rapamycin (EVEROLIMUS®), 40-O-(3-hydroxypropyl)rapamycin, 40-O-[2-(2-hydroxyethoxy)]ethyl-rapamycin, and 40-O-tetrazolylrapamycin, synthetic inorganic and organic compounds, proteins and peptides, polysaccharides and other sugars, lipids, and DNA and RNA nucleic acid sequences having therapeutic, prophylactic or diagnostic activities, nucleic acid sequences include genes, antisense molecules which bind to complementary DNA to inhibit transcription, and ribozymes. Some other examples of suitable bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy; antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents.

As mentioned previously, a dynamer of the invention may be used in a coating applied to a medical device. The coating can be a primer layer, a reservoir layer, a topcoat layer, or any combination thereof.

As used herein, a “primer layer” refers to a coating consisting of a dynamer of the present invention that exhibits good adhesion characteristics with regard to the material of which the implantable medical device body is manufactured and good adhesion characteristic with regard to whatever material is to be coated on the device body. Thus, a primer layer serves as an intermediary layer between a device body and materials to be affixed to the device body.

As used herein, “reservoir layer” refers to a dynamer layer that has dispersed within its three-dimensional structure one or more therapeutic agents. A dynamer reservoir layer is designed to release therapeutic agent into the surrounding environment by, without limitation, elution or biodegradation of the dynamer.

As used herein, “topcoat layer” refers to an outermost layer disposed over the reservoir layer and primer layer. The topcoat layer can act as a protective coating as well as a rate controlling layer.

As used herein, “disposed over” means that a layer of material, e.g., dynamers, is physically present over a device or another layer. The layer can be formed by any means presently known, or as such may become known in the future including at present, without limitation, spraying, dipping, electrodeposition, roll coating, brushing, direct droplet application and molding.

The use of dynamers in medical device coatings, as provided by the present invention, provides novel means for controlling the release of therapeutic agents at a disease locale.

Another aspect of the present invention relates to a method of treating or preventing a vascular disease. The vascular disease may be, without limitation, atherosclerosis, restenosis, vulnerable plaque or peripheral arterial disease and the method involves providing an implantable medical device of the present invention and implanting the medical device in a vessel of a patient.

As used herein, a “patient” refers to any organism that can benefit from the administration of a therapeutic agent. In particular, patient refers to a mammal such as a cat, dog, horse, cow, pig, sheep, rabbit, goat or a human being.

As used herein, “treating” refers to the administration of a therapeutically effective amount of a therapeutic agent to a patient known or suspected to be suffering from a vascular disease.

As used herein, “known” to be afflicted with a vascular disease refers first to a condition that is relatively readily observable and or diagnosable. An example, without limitation, of such a disease is atherosclerosis, which is a discrete narrowing of a patient's arteries. Restenosis, on the other hand, while in its latter stages, like atherosclerosis, is relatively readily diagnosable or directly observable, may not be so in its nascent stage. Thus, a patient may be “suspected” of being afflicted or of being susceptible to affliction with restenosis at some time subsequent to a surgical procedure to treat an atherosclerotic lesion. Further, while restenosis tends generally to occur at the same locus as a previous atherosclerotic lesion, it may not be exactly so, so a region of a segment of a vessel somewhat distant from the site of the initial atherosclerosis may in fact be the site of restenosis.

An atherosclerotic lesion refers to a deposit of fatty substances, cholesterol, cellular waste products, calcium and/or fibrin on the inner lining or intima of an artery.

Restenosis refers to the re-narrowing or blockage of an artery at or near the site where angioplasty or another surgical procedure was previously performed to remove a stenosis.

Vulnerable plaque on the other hand is quite different from either atherosclerosis or restenosis and would generally come under the designation “suspected” affliction. This is because vulnerable plaque occurs primarily within the wall of a vessel and does not cause prominent protrusions into the lumen of the vessel. It is often not until it is “too late,” i.e., until after a vulnerable plaque has broken and released its components into the vessel, that its presence is even known. Numerous methods have and are being investigated for the early diagnosis of vulnerable plaque but to date none have proven completely successful. Thus, the regional treatment of a segment of a vessel suspected of being afflicted with vulnerable plaque may be the best way to address such lesions.

As used herein, a peripheral arterial disease refers to a condition similar to coronary artery disease and carotid artery disease in which fatty deposits build up in the inner linings of the artery walls thereby restricting blood circulation, mainly in arteries leading to the kidneys, stomach, arms, legs and feet.

Methods of implanting a medical device in a vessel are known to those skilled in the art.

Another aspect of the present invention relates to a composition that includes a dynamer and a therapeutic agent encapsulated or embedded within the dynamer.

As used herein, “encapsulated within” means the therapeutic agent is contained within a dynamer structure.

As used herein, “embedded within” means the therapeutic agent is integrated into the backbone of the dynamer structure.

Suitable dynamers and suitable therapeutic agents are described above.

In various embodiments, the dynamer is used to make particles, hydrogel particles, polymer depots or hydrogel depots.

As used herein, “particle” refers to a solid matrix, porous structure or shell structure. The particle can be any shape and size.

As used herein, “hydrogel particle” refers to a cross-linked network of polymer chains that is absorbent but stable in an aqueous environment. Hydrogel particles can be used to encapsulate therapeutic agents by methods known to those skilled in the art.

As used herein, “polymer depot” refers to polymer particles that make up a matrix capable of containing a bioactive agent. The matrix can be any shape and size. The matrix can be injectable.

As used herein, “hydrogel depot” refers to hydrogel particles that make up a matrix capable of containing a bioactive agent. The matrix can be any shape and size. The matrix can be injectable.

Therapeutic agents that can be encapsulated or embedded within the polymer are described above. Methods of encapsulating or embedding agents in a polymer are known to those skilled in the art.

Another aspect of the present invention relates to a method that involves providing a composition of the present invention and administering a therapeutically effective amount of the composition to a disease locale in a patient. The disease locale can be the vasculature and the vascular disease can be atherosclerosis, restenosis, vulnerable plaque or peripheral arterial disease.

In various aspects, administering the composition to the disease locale can include intravenous, intraarterial, intraadventitial, intraperiadvential, intramyocardial, subcutaneous, intramuscular, intra-organ, intra-tumor, or subxyphoid delivery of the composition. These methods of administration are known to those skilled in the art.

In one embodiment, administering the composition to the disease locale includes using a device. The device can be, without limitation, a catheter, an implantable medical device or a stent.

As used herein, a “therapeutically effective amount” refers to the amount of therapeutic agent that has a beneficial effect, which may be curative or palliative, on the health and well-being of a patient with regard to a vascular disease with which the patient is known or suspected to be afflicted. A therapeutically effective amount may be administered as a single bolus, as intermittent bolus charges, as short, medium or long term sustained release formulations or as any combination of these.

The amount of therapeutic agent will depend on the required minimum effective concentration (MEC) of the agent and the length of time over which it is desired that the MEC be maintained. For most therapeutic agents the MEC will be known to, or readily derivable by, those skilled in the art from the literature. For experimental therapeutic agents or those for which the MEC by localized delivery is not known, such can be empirically determined using techniques well-known to those skilled in the art.

As used herein, “disease locale” refers to any location within a patient's body where abnormal physiological conditions exist.

As used herein, “vascular disease locale” refers to the location within a patient's body where an atherosclerotic lesion(s) is present, where restenosis may develop, the site of vulnerable plaque(s) or the site of a peripheral arterial disease.

The present invention provides a system that uses dynamers for use in medical device coatings and particle compositions, and further provides methods of using such to treat diseases, such as, without limitation vascular diseases. One such system is described in detail above, i.e., polyacylhydrazones, although other systems using imines and oximes are also encompassed by the present invention. The dynamic nature of these assemblies provides novel drug delivery device coatings and drug encapsulating components, thereby fulfilling a need for better and more efficacious ways of treating vascular disease.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. An implantable medical device comprising a dynamer.

2. The implantable medical device according to claim 1, wherein the dynamer comprises covalent bonds that are reversible under physiological conditions.

3. The implantable medical device according to claim 2, wherein the reversible covalent bonds comprise hydrazones, imines, oximes, or a combination thereof.

4. The implantable medical device according to claim 1, wherein the device is a stent.

5. The implantable medical device according to claim 1, wherein the device comprises a therapeutic agent.

6. The implantable medical device according to claim 5, wherein the therapeutic agent is selected from the group consisting of an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant.

7. The implantable medical device of claim 1, wherein the dynamer further comprises reversible non-covalent interactions.

8. The implantable medical device according to claim 7, wherein the reversible non-covalent interactions are reversible under physiological conditions.

9. The implantable medical device according to claim 8, wherein the reversible non-covalent interactions comprise hydrogen bonding, ionic bonding, chelation, charge-transfer complexes, pi-stacking, hydrophobic interactions, electrostatic interactions, magnetic interactions or van der Waals forces.

10. A coating for a medical device comprising a dynamer.

11. The coating according to claim 10, wherein the dynamer comprises covalent bonds that are reversible under physiological conditions.

12. The coating according to claim 11, wherein the reversible covalent bonds comprise hydrazones, imines, oximes, or a combination thereof.

13. The coating of claim 10, wherein the dynamer further comprises reversible non-covalent interactions.

14. The coating according to claim 13, wherein the reversible non-covalent interactions are reversible under physiological conditions.

15. The coating according to claim 14, wherein the reversible non-covalent interactions comprise hydrogen bonding, ionic bonding, chelation, charge-transfer complexes, pi-stacking, hydrophobic interactions, electrostatic interactions, magnetic interactions or van der Waals forces.

16. The coating according to claim 10, wherein the coating comprises a primer layer, a reservoir layer, a topcoat layer, or any combination thereof.

17. A method of treating or preventing a vascular disease comprising:
providing an implantable medical device according to claim 1; and
implanting the medical device in a vessel of a patient in need thereof.

18. The method according to claim 17, wherein the vascular disease comprises atherosclerosis, restenosis, vulnerable plaque or peripheral arterial disease.

19. A composition comprising:
a dynamer; and
a therapeutic agent encapsulated or embedded within the dynamer.

20. The composition according to claim 19, wherein the dynamer comprises covalent bonds that are reversible under physiological conditions.

21. The composition according to claim 20, wherein the reversible covalent bonds comprise hydrazones, imines, oximes, or a combination thereof.

22. The composition according to claim 19, wherein the dynamer comprises particles, hydrogel particles, a polymer depot or a hydrogel depot.

23. The composition according to claim 19, wherein the therapeutic agent is selected from the group consisting of an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant.

24. The composition of claim 19, wherein the dynamer further comprises reversible non-covalent interactions.

25. The composition according to claim 24, wherein the reversible non-covalent interactions are reversible under physiological conditions.

26. The composition according to claim 25, wherein the reversible non-covalent interactions comprise hydrogen bonding, ionic bonding, chelation, charge-transfer complexes, pi-stacking, hydrophobic interactions, electrostatic interactions, magnetic interactions or van der Waals forces.

27. A method comprising:
providing a composition according to claim 19; and
administering a therapeutically effective amount of the composition to a disease locale in a patient.

28. The method according to claim 27, wherein the disease locale is a vascular disease locale.

29. The method according to claim 27, wherein administering the composition to the disease locale comprises intravenous, intraarterial, intraadventitial, intraperiadvential, intramyocardial, subcutaneous, intramuscular, intra-organ, intra-tumor, or subxyphoid delivery of the composition.

30. The method according to claim 29, wherein administering the composition to the disease locale comprises using a device.

31. The method according to claim 30, wherein the device comprises a catheter.

32. The method according to claim 31, wherein the catheter comprises an implantable medical device.

33. The method according to claim 32, wherein the implantable medical device comprises a stent.

34. The method according to claim 28, wherein the vascular disease comprises atherosclerosis, restenosis, vulnerable plaque or peripheral arterial disease.
